(19) United States
(12) Patent Application Publication
(10) Pub. No.: US 2004/0197339 A1
(43) Pub. Date: Oct. 7, 2004

(54) Vaccine for Lyme disease

(76) Inventor: Paul R. Brown, New York, NY (US)

Correspondence Address: WIGGIN AND DANA LLP, ATTENTION: PATENT DOCKETING, ONE CENTURY TOWER, P.O. BOX 1832, NEW HAVEN, CT, 06508-1832, US

(21) Appl. No.: 10/756,245

(22) Filed: Jan. 13, 2004

Related U.S. Application Data
(60) Provisional application No. 60/443,344, filed on Jan. 29, 2003.

(51) Int. Cl. A61K 39/40; A61K 35/16

(52) U.S. Cl. 424/164.1; 424/531

(57) ABSTRACT

The present invention is directed to a pharmaceutical composition for the treatment and/or prevention of Lyme disease in a patient, comprising serum of deer blood comprising Borrelia burgdorferi bacteria and antibodies thereto, wherein the amount of the Borrelia burgdorferi bacteria is at a level that does not cause infection of the patient.

### CROSS-REFERENCE FOR RELATED APPLICATIONS

This application claims the benefit of U.S. Provisional Application No. 60/443,344, filed on Jan. 29, 2003, the disclosure of which is incorporated by reference in its entirety.

### BACKGROUND OF THE INVENTION

1. Field of the Invention

The present invention is directed to vaccines for Lyme disease, and more particularly to vaccines for Lyme disease that include antibodies derived from deer blood.

2. Brief Description of the Related Art

Lyme disease (Lyme borreliosis) is a bacterial infection spread by certain kinds of ticks. Lyme disease itself is caused by infection with Borrelia burgdorferi (B. burgdorferi) bacteria. In different parts of the United States, different kinds of ticks carry the bacteria that can cause Lyme disease. Deer ticks spread Lyme disease in the northeastern and upper midwestern United States. Western black-legged ticks spread the disease on the Pacific coast (mostly Northern California and Oregon). The ticks that spread Lyme disease are very small (about the size of a poppy seed or sesame seed), and their bite is usually painless.

If a person is bitten by a tick carrying Lyme disease bacteria, a rash often develops at the site of the tick bite within 1 to 31 days. The rash (which may look like a bull's-eye) slowly expands and may become very large. Flu-like symptoms may also occur. This early stage of the disease is called early localized Lyme disease. Lyme disease develops in three stages. If Lyme disease is not detected and treated properly during the early localized stage, the infection may progress to the second or third stages of Lyme disease and involve the skin, joints, nervous system, and heart. The second stage of Lyme disease, called early disseminated Lyme disease, may develop several weeks or months after a person becomes infected. It can cause skin problems, joint problems, early nervous system problems, and heart problems. The last stage of the disease, called late persistent Lyme disease, is often the most serious and may develop weeks, months, or, on rare occasion, even years after the initial infection. It can cause joint problems, late nervous system problems, and heart problems.

Lyme disease may be difficult to diagnose because its symptoms are similar to those of many other illnesses. The early, often vague flu-like symptoms can easily be mistaken for another illness, especially when the typical rash of Lyme disease does not occur with them. Later symptoms of untreated Lyme disease, such as joint problems, weakness or numbness in the arms or legs, severe fatigue, or difficulties with memory and thinking, may resemble other forms of arthritis, fibromyalgia, chronic fatigue syndrome, multiple sclerosis, and other conditions.

Lyme disease is treated with antibiotics. A recent study found that if a single dose of the antibiotic doxycycline is given within 72 hours after being bitten by an infected tick, the chances of developing Lyme disease can be reduced by as much as 87% (Nadelman RB (2001). Prophylaxis with single-dose doxycycline for the prevention of Lyme disease after an Ixodes scapularis tick bite. New England Journal of Medicine, 345(2)).

It would be desirable to prevent contraction of Lyme disease rather than treating its symptoms after infection has taken place. A Lyme disease vaccination called LYMErix (SmithKline Beecham) was available for people in high-risk areas. The key ingredient in LYMErix was a genetically engineered protein from the surface of the bacteria B. Burgdorferi that helps stimulate an immune response against the bacteria. The protein, called OspA, stimulates antibodies that disable B. burgdorferi bacteria's ability to infect people. However, OspA triggers autoimmune arthritis in some individuals. Consequently, LYMErix was recently removed from the market.

Treatments for Lyme disease are the subject of several U.S. patents. U.S. Pat. No. 6,486,130 to Livey, et al. discloses immunogenic formulations comprising different serological forms of OspC to retard or prevent the development of Lyme disease. U.S. Pat. No. 6,368,603 to Jarecki-Black discloses compositions containing a Borrelia burgdorferi antigen that are useful for eliciting an immunological response in a host mammal susceptible to Lyme disease. U.S. Pat. No. 6,303,129 to Alliger, et al. discloses a process for the preparation of a vaccine from substantially viable Borrelia burgdorferi bacteria, and being capable of inducing an immune or therapeutic response against Lyme Disease when administered to a patient.

Given the severity and widespread nature of Lyme disease, what is needed in the art is a vaccine to prevent Lyme disease that is simple to prepare and administer to patients. The present invention is believed to be an answer to that need.

### SUMMARY OF THE INVENTION

In one aspect, the present invention is directed to a pharmaceutical composition for the treatment and/or prevention of Lyme disease in a patient, comprising serum of deer blood comprising Borrelia burgdorferi bacteria and antibodies thereto, wherein the amount of the Borrelia burgdorferi bacteria is at a level that does not cause infection of the patient.

In another aspect, the present invention is directed to a pharmaceutical composition for the treatment and/or prevention of Lyme disease in a patient, comprising antibodies to Borrelia burgdorferi bacteria.

In another aspect, the present invention is directed to methods of treating or preventing Lyme disease in a patient, comprising the step of administering to said patient the above pharmaceutical compositions.

These and other aspects will become apparent upon reading the following detailed description of the invention.

### DETAILED DESCRIPTION OF THE INVENTION

It has now been found that a vaccine for Lyme disease may be prepared from deer blood. It is known that deer do not contract Lyme disease, and it is believed that the antibodies contained in deer blood are effective at combating the bacteria that cause Lyme disease.

As indicated above, the present invention is a pharmaceutical composition for the treatment and/or prevention of Lyme disease in a patient, comprising serum of deer blood, comprising Borrelia burgdorferi bacteria and antibodies thereto, wherein the amount of the Borrelia burgdorferi bacteria is at a level that does not cause infection of said patient. Each of these components is discussed in more detail below.

In accordance with the present invention, deer blood infected with Borrelia burgdorferi bacteria is used as the source of antibodies for Lyme disease. Deer blood may be collected from captured animals, and removed in any appropriate quantity (typically 5 to 100 ml). Following collection of the blood, erythrocytes, neutrophils, and other large particles are removed from the blood by centrifugation or other technique, and serum is isolated. The serum contains, among other things, antibodies to Borrelia burgdorferi bacteria, as well as the Borrelia burgdorferi bacteria itself.

The collected serum is next tested for the quantity (titer) of Borrelia burgdorferi bacteria. In general, the titer of bacteria in the serum should not be so great as to cause infection of the patient when administered; however, the titer of bacteria is preferably at a level such that the immune system of the patient begins producing its own antibodies to the bacteria. The appropriate level of Borrelia burgdorferi bacteria in the serum that achieves the above results may be determined by trial and error procedures using model animals such as dogs. If the titer of Borrelia burgdorferi bacteria is too high, dimethylsulfoxide (DMSO), molecular oxygen, or pau d'arco (also known as lepacho) may be added to the serum to reduce the level of bacteria.

Alternatively, the pharmaceutical composition of the invention may include only antibodies to the Borrelia burgdorferi bacteria. In this embodiment, the antibodies may be isolated from the collected deer serum using conventional chromatography techniques (e.g., preparative affinity chromatography) well known in the art.

The compositions of the invention are preferably administered internally, e.g., intravenously, in the form of conventional pharmaceutical preparations, for example in conventional enteral or parenteral pharmaceutically acceptable excipients containing organic and/or inorganic inert carriers, such as water, gelatin, lactose, starch, magnesium stearate, talc, plant oils, gums, alcohol, Vaseline, or the like. The pharmaceutical preparations can be in conventional solid forms, for example, tablets, dragees, suppositories, capsules, or the like, or conventional liquid forms, such as suspensions, emulsions, or the like. If desired, they can be sterilized and/or contain conventional pharmaceutical adjuvants, such as preservatives, stabilizing agents, wetting agents, emulsifying agents, buffers, or salts used for the adjustment of osmotic pressure. The pharmaceutical preparations may also contain other therapeutically active materials.

The pharmaceutical preparation of the invention should include an amount of the compound of the invention effective for treating or preventing Lyme disease. The effective dosage will depend on the activity of the antibodies employed and is thus within the ordinary skill of the art to determine for any particular host mammal or other host organism. Suitable dosages may be, for example, in the range of about 0.5-15 mg per kg for a human being.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entireties.

What is claimed is:

1. A pharmaceutical composition for the treatment and/or prevention of Lyme disease in a patient, comprising;
serum of deer blood comprising Borrelia burgdorferi bacteria and antibodies thereto, wherein the amount of said Borrelia burgdorferi bacteria is at a level that does not cause infection of said patient.

2. The pharmaceutical composition of claim 1, further comprising dimethylsulfoxide (DMSO).

3. The pharmaceutical composition of claim 1, further comprising molecular oxygen.

4. The pharmaceutical composition of claim 1, further comprising pau d'arco.

5. The pharmaceutical composition of claim 1, further comprising a pharmaceutically acceptable carrier.

6. A pharmaceutical composition for the treatment and/or prevention of Lyme disease in a patient, comprising antibodies to Borrelia burgdorferi bacteria.

7. The pharmaceutical composition of claim 6, further comprising dimethylsulfoxide (DMSO).

8. The pharmaceutical composition of claim 6, further comprising molecular oxygen.

9. The pharmaceutical composition of claim 6, further comprising pau d'arco.

10. The pharmaceutical composition of claim 6, wherein said antibodies are derived from deer.

11. The pharmaceutical composition of claim 6, further comprising a pharmaceutically acceptable carrier.

12. A method of treating or preventing Lyme disease in a patient, comprising the step of administering to said patient the pharmaceutical composition of claim 1 or claim 6.
